(19) United States
(12) Patent Application Publication
(10) Pub. No.: US 2004/0019959 A1
(43) Pub. Date: Feb. 5, 2004

(54) Dive mask with integral side mounted rear view mirrors

(76) Inventor: Walter L. Bloom JR., Atlanta, GA (US)

Correspondence Address: Walter L. Bloom, Jr., 841 Peachtree Hills Circle, Atlanta, GA, 30305-4249, US

(21) Appl. No.: 10/211,730

(22) Filed: Aug. 2, 2002

(51) Int. Cl. G02C 1/00; A61F 9/02

(52) U.S. Cl. 2/428; 351/43

(57) ABSTRACT

A dive mask with front-view and side-view lenses and with integral side mounted rear view mirrors.

### TECHNICAL FIELD

This device relates generally to equipment for use principally in scuba diving, and more particularly to dive masks.

### BACKGROUND OF THE INVENTION

Many styles and models of scuba diving masks exist, and many recent designs attempt to maximize the overall field of vision of their users. To accomplish this objective, mask designers typically vary the sizes, shapes and numbers of lenses.

Of course, visual information originating from events that occur to the rear of the diver also may be of great value. For example, such information may assist a diver in locating dive buddies and in identifying the approach of potential threats such as sharks, as well as in more easily identifying overhead obstructions and sources of entanglements. Not surprisingly, therefore, several U.S. patents currently exist for devices that extend a diver's field of vision to events that occur behind the diver.

In particular, U.S. Pat. No. 5,216,454 granted to Berke incorporates optical prisms that are permanently attached to the front lens of a dive mask. Since the prisms occupy a portion of a diver's forward-facing field of vision, they limit said field of vision while providing potentially distracting information that may compete with a diver's attention to information coming from said forward-facing field of vision.

In contrast, U.S. Pat. No. 5,914,769 granted to Varrichone employs a convex mirror pivotally mounted on an extended arm that attaches to a clamping device so that the invention can be used with most conventionally designed dive masks. Visual information that is supplied by this device is somewhat limited to that originating on one side of a diver's body. Furthermore, the device may need to be manipulated to adjust its viewing angle from time to time during a dive. Since it projects as a separate object in front of a mask, it also is prone to being struck by external objects and to becoming entangled in lines and hoses. As a consequence, a diver may have to attend to the device excessively and at inopportune moments while diving.

Consequently, a need yet remains for a rear-view device for dive masks that a) provides as much information as possible concerning events that may occur to the rear of a diver's forward-facing field of vision, b) does not compete with a diver's attention to information coming from said forward-facing field of vision, and c) imposes as little inconvenience and as few additional requirements as possible during a dive.

### SUMMARY OF THE INVENTION

Briefly described, in a first preferred embodiment, the present invention comprises a dive mask with side-view as well as front-view lenses and with a separate rear-view mirror integrally and nondetachably mounted to each side of the frame of the mask so as to reflect light through one side-view lens, said light originating from behind the forward facing field of vision of the mask.

In a second preferred embodiment, the present invention comprises a dive mask with side-view as well as front-view lenses and with a separate rear-view mirror integrally but detachably mounted to each side of the frame of the mask so as to reflect light through one side-view lens, said light originating from behind the forward facing field of vision of the mask.

In order to reflect light from each side mounted rear view mirror through one of the mask's side lenses, the angle of attachment of each such mirror to the mask's frame for both preferred embodiments extends from a line making roughly a 15 degree angle with a line drawn parallel to the forward facing plane of the mask to a line making roughly a 45 degree angle with said parallel line.

### BRIEF DESCRIPTIONS OF THE DRAWINGS

FIG. 1 shows a front view of a dive mask with front-view and side-view lenses.

FIG. 2 shows a top view of a dive mask with front-view and side-view lenses.

FIG. 3 shows a left side view of a dive mask with front-view and side-view lenses.

FIG. 4 shows a front view of a modified version of the mask in FIG. 1 with the addition of integral side mounted rear view mirrors.

FIG. 5 shows a top view of a modified version of the mask in FIG. 2 with the addition of integral side mounted rear view mirrors.

FIG. 6 shows a left side view of a modified version of the mask in FIG. 3 with the addition of integral side mounted rear view mirrors.

### DETAILED DESCRIPTIONS OF THE PREFERRED EMBODIMENTS

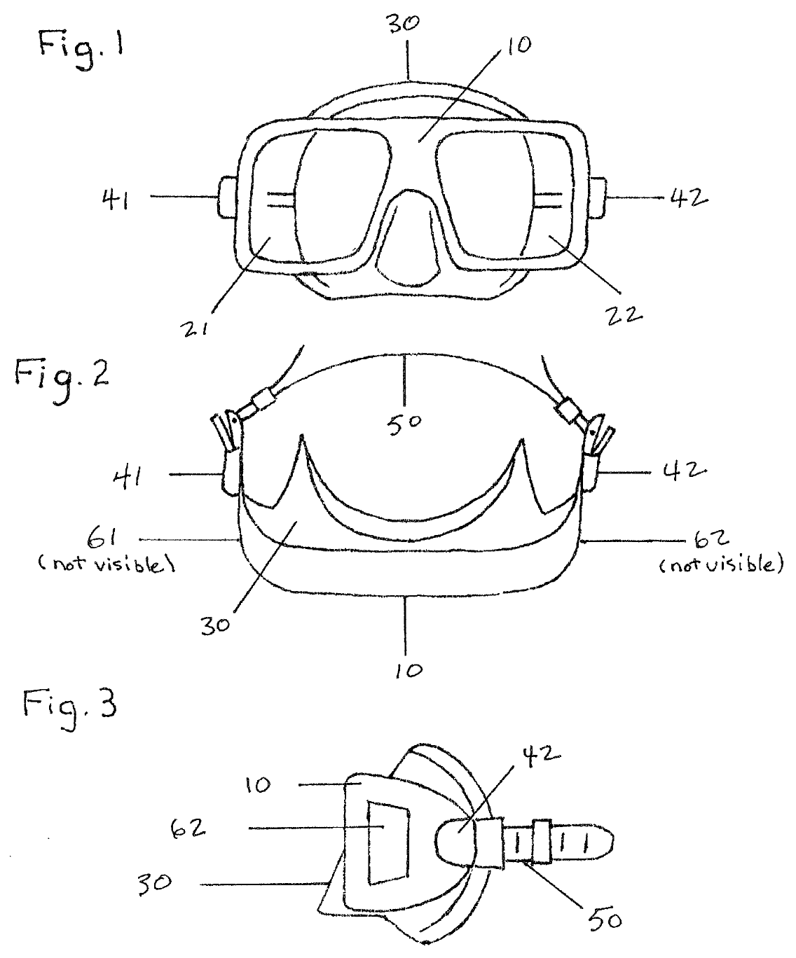

FIGS. 1, 2 and 3 respectively show front, top and left side views of a dive mask with both front-view and side-view lenses. FIG. 1 shows a front view of the mask. The mask frame 10 holds the two front-view lenses 21 and 22 as well as the silicon rubber skirt 30 in watertight enclosures. Strap attachment devices 41 and 42, by means of which head strap 50 (shown in FIG. 2) is adjustably attached to the frame 10, also are permanently connected to the left and right sides of the exterior of the frame 10. In addition to showing the position of head strap 50, the top view of the mask in FIG. 2 indicates the lateral positions of right and left side-view lenses 61 and 62, respectively. The left side view of the mask in FIG. 3 then shows the relationship between left side-view lens 62 and the frame 10 within which it is sealed.

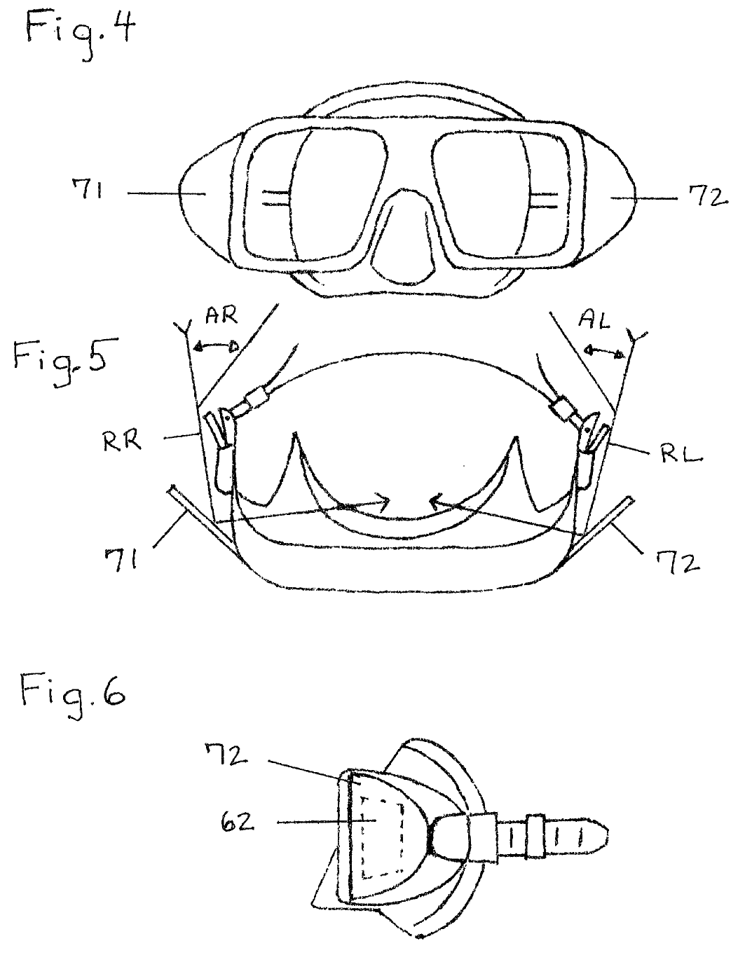

FIGS. 4, 5 and 6 respectively show front, top and left side views of the device—namely a dive mask with both front-view and side-view lenses and with integral side mounted rear view mirrors. The front view in FIG. 4 shows the external non-reflective surfaces of the two mirrors 71 and 72 that are attached on the right and the left side of the frame 10, respectively. The top view in FIG. 5 shows rays of light RR and RL reflecting off of the internal reflective surfaces of mirrors 71 and 72, respectively. Said rays RR and RL reflect through side-view lens 61 and 62, respectively, and are shown originating from angles of less than 180 degrees relative to the mask's forward facing line of vision. Nevertheless, angles AR and AL reveal that a diver can use mirrors 71 and 72 to see objects not only straight back but also directly behind simply by turning his or her head through angles ranging roughly from 0 to 45 degrees to either the left or the right. Finally, FIG. 6 shows a view of the left side mounted rear view mirror 72 in relation to left side view lens 62.

I claim:

1. A dive mask with side-view as well as front-view lenses and with a separate rear-view mirror integrally and nondetachably mounted to each side of the frame of the mask so as to reflect light through one side-view lens, said light originating from behind the forward facing field of vision of the mask.

2. A dive mask with side-view as well as front-view lenses and with a separate rear view mirror integrally but detachably mounted to each side of the frame of the mask so as to reflect light through one side-view lens, said light originating from behind the forward facing field of vision of the mask.

3. A device as claimed in claim 1 wherein the possible angle of attachment of a rear view mirror to the frame of the device extends from a line making roughly a 15 degree angle with a line drawn parallel to the forward facing plane of the mask to a line making roughly a 45 degree angle with said parallel line.

4. A device as claimed in claim 2 wherein the possible angle of attachment of a rear view mirror to the frame of the device extends from a line making roughly a 15 degree angle with a line drawn parallel to the forward facing plane of the mask to a line making roughly a 45 degree angle with said parallel line.
